(19) United States
(12) Patent Application Publication
(10) Pub. No.: US 2004/0015086 A1
(43) Pub. Date: Jan. 22, 2004

(54) Apparatus capable of accommodating a sphygmomanometer and medicine

(75) Inventor: Paul Yang, Chung-Ho City (TW)

Correspondence Address: Finnegan, Henderson, Farabow,, Garrett & Dunner, L.L.P., 1300 I Street, N.W., Washington, DC, 20005-3315, US

(73) Assignee: HEALTH & LIFE CO., LTD.

(21) Appl. No.: 10/197,420

(22) Filed: Jul. 18, 2002

(51) Int. Cl. A61B 5/02

(52) U.S. Cl. 600/490

(57) ABSTRACT

In an apparatus capable of accommodating a sphygmomanometer and medicine, a housing is formed with a first receiving space adapted for accommodating the sphygmomanometer therein, and a second receiving space adapted for accommodating the medicine therein. An operating unit is mounted on the housing and is manually operable so as to generate input signals. A timing unit is mounted in the housing and is coupled to the operating unit for receiving the input signals therefrom. The timing unit is capable of serving as a clock, and sets predetermined time information in accordance with the input signals from the operating unit. A display unit is mounted on the housing, and is coupled to and controlled by the timing unit for showing time information thereon.

### BACKGROUND OF THE INVENTION

1. Field of the Invention

The invention relates to an apparatus for accommodating a sphygmomanometer, more particularly to an apparatus capable of accommodating a sphygmomanometer and medicine.

2. Description of the Related Art

People with abnormal blood pressure usually have to frequently monitor their blood pressure by means of a sphygmomanometer. In addition, medicine has to be taken periodically. Usually, a sphygmomanometer and medicine are separately disposed. As such, it is inconvenient for hypertensive people to carry a sphygmomanometer and medicine when traveling.

### SUMMARY OF THE INVENTION

Therefore, the object of the present invention is to provide an apparatus capable of accommodating a sphygmomanometer and medicine.

According to the present invention, an apparatus is capable of accommodating a sphygmomanometer and medicine and comprises:

a housing formed with a first receiving space adapted for accommodating the sphygmomanometer therein, and a second receiving space adapted for accommodating the medicine therein;

an operating unit mounted on the housing and manually operable so as to generate input signals;

a timing unit mounted in the housing and coupled to the operating unit for receiving the input signals therefrom, the timing unit being capable of serving as a clock and setting predetermined time information in accordance with the input signals from the operating unit; and

a display unit mounted on the housing and coupled to and controlled by the timing unit for showing time information thereon.

The timing unit further generates an alarm output when current time information shown on the display unit matches the predetermined time information.

### BRIEF DESCRIPTION OF THE DRAWINGS

Other features and advantages of the present invention will become apparent in the following detailed description of the preferred embodiments with reference to the accompanying drawings, of which:

FIG. 1 is a perspective view showing the first preferred embodiment of an apparatus capable of accommodating a sphygmomanometer and medicine according to the present invention;

FIG. 2 is a perspective view showing the first preferred embodiment in an opened state; and

FIG. 3 is a perspective view showing the second preferred embodiment of an apparatus capable of accommodating a sphygmomanometer and medicine according to the present invention.

### DETAILED DESCRIPTION OF THE PREFERRED EMBODIMENTS

Before the present invention is described in greater detail, it should be noted that like elements are denoted by the same reference numerals throughout the disclosure.

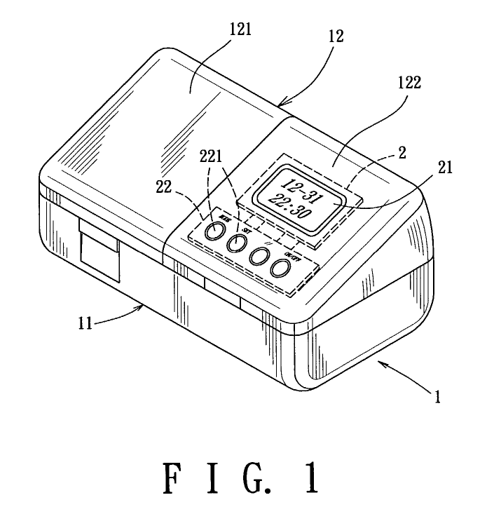

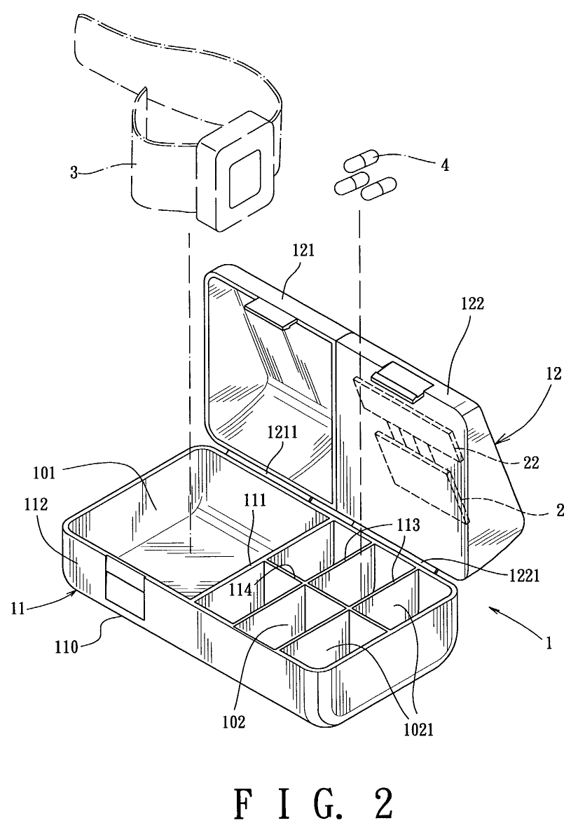

Referring to FIGS. 1 and 2, the first preferred embodiment of an apparatus 1 according to the present invention is shown to include a housing 1, an operating unit 22, a timing unit 2, and a display unit 21.

The housing 1, which may be formed from plastic material by injection molding, includes abase body 11, and a cover body 12 connected pivotally to the base body 11. The cover body 12 includes first and second cover members 121, 122. The first and second cover member 121,122 are connected pivotally to the base body 11 via first and second pivot portions 1211, 1221, respectively, such that the first and second cover member 121, 122 are pivotable relative to the base body 11. The base body 11 has a bottom wall 110, a surrounding wall 112 extending upwardly from a periphery of the bottom wall 111, and an inner wall 111 disposed on the bottom wall 110 and having opposite ends connected to the surrounding wall 112 such that the first cover member 121 and a part of the surrounding wall 112 and the bottom wall 110 confine a first receiving space 101 that is adapted for accommodating a sphygmomanometer 3 therein, and such that the second cover member 121 and the remainder of the surrounding wall 112 and the bottom wall 110 confine a second receiving space 102 that is adapted for accommodating medicine 4 therein. The base body 11 further has a plurality of partition walls 113, 114 provided in the second receiving space 102 so as to divide the second receiving space 102 into a plurality of compartments 1021 adapted for accommodating various kinds of medicine 4.

The operating unit 22, which includes a plurality of touch-control keys 221, is mounted on the second cover member 122 of the cover body 12 of the housing 1, and is manually operable so as to generate input signals.

The timing unit 2 is mounted in the second cover member 122 of the cover body 12 of the housing 1, and is coupled to the operating unit 22 for receiving the input signals therefrom. The timing unit 2 is capable of serving as a clock, and sets predetermined time information in accordance with the input signals from the operating unit 22. In practice, the predetermined time information may be decided in accordance with times for taking medication or measuring blood pressure. Since the specific configuration of the timing unit 2 is known in the art and is not critical to the present invention, a detailed description of the same will be omitted herein.

The display unit 21, such as a liquid crystal display module, is mounted on the second cover member 122, and is coupled to and controlled by the timing unit 2 for showing time information thereon.

It is noted that the timing unit 2 further generates an alarm output when current time information shown on the display unit 21 matches the predetermined time information. In this embodiment, the alarm output is an audio output so as to remind the user when to take medicine or measure blood pressure.

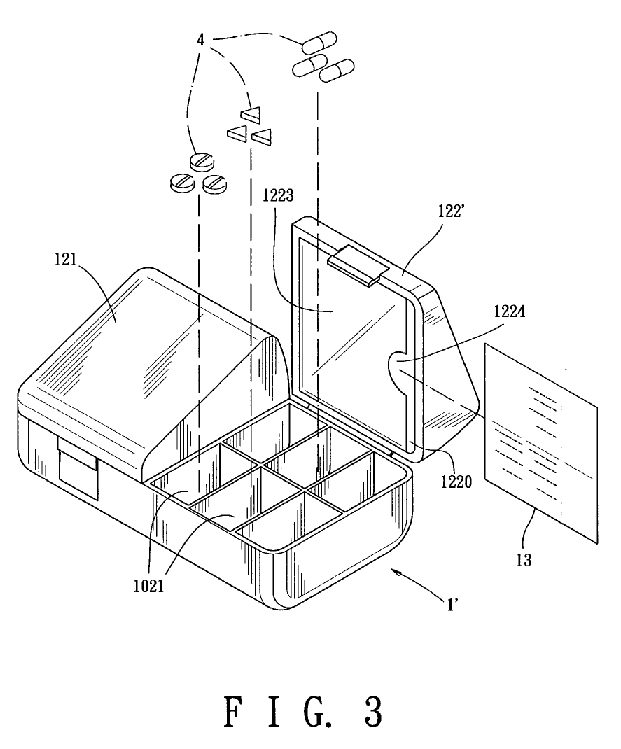

FIG. 3 illustrates the second preferred embodiment of an apparatus 1′ capable of accommodating a sphygmomanometer (not shown) and medicine 4, which is a modification of the first preferred embodiment. Unlike the previous embodiment, the second cover member 122′ has an inner 1220 with a transparent sheet 1223 attached thereto to form a card pocket that is adapted to receive a card 13 therein. The card 13 may be printed with name of the medicine 4 in the compartments 1021 and dosage information for reference when taking the medicine 4. The transparent sheet 1223 is formed with a notch 1224 to facilitate insertion of the card 13 into the card pocket.

While the present invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

I claim:

1. An apparatus capable of accommodating a sphygmomanometer and medicine, comprising:
a housing formed with a first receiving space adapted for accommodating the sphygmomanometer therein, and a second receiving space adapted for accommodating the medicine therein;
an operating unit mounted on said housing and manually operable so as to generate input signals;
a timing unit mounted in said housing and coupled to said operating unit for receiving the input signals therefrom, said timing unit being capable of serving as a clock and setting predetermined time information in accordance with the input signals from said operating unit; and
a display unit mounted on said housing and coupled to and controlled by said timing unit for showing time information thereon;
said timing unit further generating an alarm output when current time information shown on said display unit matches the predetermined time information.

2. The apparatus as claimed in claim 1, wherein said housing includes a base body, and a cover body connected pivotally to said base body and provided with said operating unit, said timing unit and said display unit.

3. The apparatus as claimed in claim 2, wherein said cover body includes first and second cover members, and said base body has a bottom wall, a surrounding wall extending upwardly from a periphery of said bottom wall, and an inner wall disposed on said bottom wall and having opposite ends connected to said surrounding wall such that said first cover member and a part of said surrounding wall and said bottom wall confine said first receiving space and such that said second cover member and the remainder of said surrounding wall and said bottom wall confine said second receiving space.

4. The apparatus as claimed in claim 3, wherein said base body further has a plurality of partition walls provided in said second receiving space.

5. The apparatus as claimed in claim 3, wherein said second cover member has an inner surface with a transparent sheet attached thereto to form a card pocket that is adapted to receive a card therein.

6. The apparatus as claimed in claim 1, wherein the alarm output is an audio output.
